(19) United States
(12) Patent Application Publication
(10) Pub. No.: US 2019/0085007 A1
(43) Pub. Date: Mar. 21, 2019

(54) 4-methylcatechol Derivatives and Uses Thereof

(71) Applicant: Thanares GmbH, Potsdam (DE)

(72) Inventors: Hans-Michael THIEDE, Potsdam (DE); Wolfgang KEHR, Berlin (DE)

(21) Appl. No.: 15/951,277

(22) Filed: Apr. 12, 2018

Related U.S. Application Data
(63) Continuation of application No. 15/035,276, filed on Aug. 19, 2016, now abandoned.

(30) Foreign Application Priority Data
Sep. 7, 2011 (DE) 10 2011 112 496.2

(51) Int. Cl. C07H 15/203; A61K 31/7034; A61K 9/28; A61K 9/00

(52) U.S. Cl. CPC C07H 15/203; A61K 31/7034; A61K 9/0053; A61K 9/2846

(57) ABSTRACT

The invention relates to phenoxy derivatives with glycosidically bound sugar moieties, pharmaceutical compositions containing such compounds, uses of such compounds and compositions, and methods of making such compounds and pharmaceutical compositions.

### FIELD OF THE INVENTION

The invention relates to novel phenoxy derivatives with glycosidically bound sugar moieties, which are suitable for use in pharmaceutical compositions for the treatment of peripheral and autonomic neuropathies, central nervous degenerative diseases, high blood pressure, arteriosclerosis, venous insufficiency, diabetes mellitus, osteoporosis, cataract, and photoaging of the skin suitable, pharmaceutical compositions containing such compounds, uses of such compounds, and pharmaceutical compositions and methods of making such compounds.

### BACKGROUND OF THE INVENTION AND PRIOR ART

4-Methylcatechol (4-MC) is an endogenous compound occurring in very low concentrations in the human organism, and little is known about the place of formation and the regulation of the metabolism in the human tissue. It is however known that 4-MC as a product of metabolism of orally received flavonoids such as quercetin and rutin may be formed by the activity of the intestinal microbiota in the human colon.

In in-vitro investigations and in animal experiments, the effects of 4-MC have been described, which suggest a preventive and/or therapeutic effect for humans. In the following, the different effects and the respective therapeutic applications derived therefrom are described:
1. Anti-inflammatory effects
2. Peroxyl radical and superoxide anion radical scavengers
3. Sequestrants with metal ions
4. Stimulation of the neurotrophins “nerve growth factor” (NGF), “brain derived nerve growth factor” (BDNF), “glia derived nerve growth factor” (GDNF), and others
5. Inhibition of the cholesterol biosynthesis
6. Inhibition of the lipoxygenase
7. Stimulation of the heme oxygenase
8. Inhibition of the “angiotensin converting enzyme” (ACE) activity

Certain cascade-like dependencies have been described between these different effects. 4-MC stimulates the phosphoinositol-3-kinase/AKT and the Nrf2-ARE signal transduction pathway and thereby activates the expression of the heme oxygenase, which in turn, also as a response to oxidative stress, promotes the generation of bilirubin and carbon monoxide, besides iron, which in turn promote the expression of the neurotrophins BDNF and GDNF in neurons and also glial cells (Furukawa Y. et al., Biomedical Research 2010, 31:45-52; Hung S. Y. et al., Neuro-pharmacology 2010 February, 58:321-329). All effects described above are important in view of a neuroprotective effect and underline the performance and importance of the intestinal microbial activity.

Peripheral and Autonomic Neuropathies:

To the secondary diseases of diabetes mellitus belongs diabetic neuropathy, from which 30-50% of the diabetics are suffering and which is thus the most frequent peripheral neuropathy in the Western countries (Pittenger G., Vinik A., Exp. Diabesity Res. 2003 October-December, 4(4):277-85. Review). As a disease of the peripheral nervous system, it relates to sensory as well as motor nerves, and by the heterogeneity, the thin myelinated fibers are also concerned, besides the thick myelinated fibers. A feared complication being very impairing for concerned people is the so-called “diabetic foot” (diabetic podopathy). Therein, sensory disorders with and without circulatory disorders will develop to a clinical picture that will frequently lead to an amputation of the lower extremity. Year for year, about 30,000 amputations of the lower extremity are carried out in Germany alone because of this diagnosis (Chantelau E., Deutsches Ärzteblatt 2002, 99:A 2052-2056).

From animal experiments of diabetic rats treated with streptozotozin, it is known that the expression of neurotrophins (nerve growth factors) such as “glial cell derived neurotrophic factor” (GDNF), neurotrophin-3 and NGF in the colon is reduced. This is used for explaining the known gastrointestinal complications in diabetes patients (Liu W. et al., Auton. Neurosci. 2010, 154:79-83).

Since the beginning of the nineties, it is known that there is a series of compounds that are capable to stimulate the endogenous neurotrophin generation (Furukawa Y. et al., Biochem. Pharmacol. 1990 November 75; 40 (10):2337-42). To these substances belong the alkyl catechols and here in particular 4-MC. For 4-MC, it has been shown that neuropathies in various animal experimental models (neuropathies induced by resiniferatoxin, pyridoxin, acrylamide, and cytostaticy and also the diabetic neuropathy induced by streptozotozin) are favorably influenced by the administration of 4-MC (Hanaoka Y. et al., J. Neurol. Sci. 1994 March; 122(1):28-32). Further, the ototoxicity induced by gentamycin is antagonized by 4-MC via stimulation of NGF and other neurotrophins.

Further, in the “crush injury” model of the ischiatic nerves of the mouse, 4-MC improved the reinnervation of the skin nerves, in particular of the unmyelinated nerve fibers (Hsieh Y. L. et al., J. Neuropathol. Exp. Neurol. 2009; 68:1269-128 7).

These results suggest that alkyl catechols and their derivatives have preventive and therapeutic effects in different forms of neuropathies in humans, too, such as for
diabetic neuropathy, but also for
neuropathy induced by chemotherapy, and
neuropathy as a consequence of chronic alcohol abuse

Central Nervous Degenerative Diseases:

In Germany alone, at present more than one million people suffer from a dementia disease, some 700,000 thereof from Alzheimer's disease, a neurodegenerative disease. Every year, approximately 200,000 new dementia diseases are diagnosed, of which approximately 120,000 are the Alzheimer's type. In Germany, at present 300,000-400,000 persons are affected with Parkinson's disease. Due to the demographic aging, the prevalence of both diseases will grow. The financial consequences for the health system caused by these diseases are very serious—the costs of treatment and care of an Alzheimer patient are today approximately 40,000 € per year—and will continue to grow. Therapeutics for symptomatic treatment of Parkinson's disease are available today, and first products for improving the cognitive functions of patients with Alzheimer's disease show marginal effects, a real breakthrough with active substances retarding the progress of these neurodegenerative diseases, could however not yet be achieved, in spite of intense worldwide research.

The effects of the alkyl catechols and their metabolites indicate a clear therapeutic potential:

In addition to the already mentioned stimulation of the neurotrophins that also occurs in the central nervous system and counteracts the neurodegeneration process, signal transduction effects have been described suggesting that 4-MC has, by activation of the heme oxigenase-l-expression, neuroprotective effects, in particular against the adverse oxidative stress (Furukawa Y. et al., Biomedical Res. 2010; 31:45-52). 4-MC also stimulates the mitogen-activated protein kinase (MAPK/ERK1/2) that in turn activates the cAMPresponse element binding protein (CREB). CREB plays an important role for the nerve growth as well as for the survival of the nerve cells.

Oxidative stress is associated with the extinction of nerve cells and plays a prominent role in the pathogenesis of many chronic degenerative diseases such as Alzheimer's disease, Parkinson's disease, Huntington's disease, and the amyotrophic lateral sclerosis. A signal transduction pathway, wherein the transcription activation of protective genes is mediated by a “cis-acting element”, the so-called “antioxidant responsive element” (ARE), is of growing importance. The activation by the transcription factor NF-E2-related factor 2 (Nrf2) that binds to ARE, protects nerve cells from cell death induced by oxidative stress (Johnson J. A. et al., Ann. NY Acad. Sci. 2008; 1147:61-69). 4-MC activates Nrf2 and can act in a neuroprotective manner also via this signal transduction pathway (Satoh T. et al., Biochem. Biophys. Res. Commun. 2009; 379:537-341).

Alkyl catechols such as 4-MC have additional anti-inflammatory properties that are shown by the inhibition of the expression of the inducible NO synthase and the inhibition of the release of pro-inflammatory cytokines such as TNF from microglia, thus clear neuroprotective effects being provided. (Zheng L. T. et al., Eur. J. Pharmacol. 2008; 588:106-113). These protective effects can be used for the treatment of neurodegenerative diseases being associated with a marked activation of the microglia. Maintaining or improving cognitive functions is of great importance for demented patients, e.g., patients with Alzheimer's disease. Indications that alkyl catechols have a positive influence on cognitive performance follow from investigations by Sun M. K. et al., Neuroreport. 2008; 19:355-359). After intraventricular administration 4-MC also improved the spatial learning and memory of rats, an effect, to which apparently BDNF contributes, since a simultaneous administration of BDNF antibodies ended the effect of 4-MC.

O-Methyl-metabolites, too, of alkyl catechols such as 2-methoxy-4-ethylphenol have neuroprotective effects that can be used therapeutically for the treatment of degenerative diseases of the central nervous systems. They protect nerve cells—as has been shown for hippocampus neurons—from the excessive, neurotoxic influx of calcium ions mediated by NMDA receptors (Fukumori R. et al., J. Pharmacol. Sci. 2010; 112:273-281).

High Blood Pressure/Arteriosclerosis:

From in-vitro investigations of liver cells follows that not only quercetin, but also 4-MC inhibits the hepatocellular cholesterol synthesis in the μmol range (Glässer G. et al., Phytomedicine. 2002; 9:33-40).

The inhibition of the angiotensin converting enzymes and other metallopeptidases is described in Bormann H. et al., Pharmazie. 2000; 55:129-132).

Diabetes Mellitus:

The inhibition of the non-oxidative AGE (advanced glycation endproduct) generation by 4-MC and DOPAC was shown in Pashikanti S. et al., Free Radic. Biol. Med. 2009 Dec. 4.

Melanoma/Basalioma:

4-MC inhibits the proliferation of melanoma cells without impairing the growth of normal human epidermal melanocytes (Payton F. et al., Biochem. Pharmacol. 2011).

Osteoporosis:

Metabolites of the alkyl catechols such as 2-methoxy-4-methylphenol (creosol) and 2-methoxy-4-ethylphenol that are formed by the catalytic effect of the catechol-O-methyltransferase, prevent the osteoporosis occurring after ovariectomy of mice—an experimental model of the post-menopausal osteoporosis—likely by inhibition of the osteoblasts breaking down bone tissue in conjunction with an anti-oxidative effect of osteoblasts promoting bone growth (Moriguchi N. et al., Biochem. Pharmacol. 2007; 73:385-393). For hydroxytyrosol (3,4-dihydroxy-phenyl-ethanol) a corresponding bone-protective effect has also been described for ovariectomized rats (Puel C. et al., J. Agric. Food Chem. 2008; 56:9417-9422).

From the above follows that it would be desirable, for the prophylaxis as well as for the therapy of the mentioned diseases, to have means, which ensure a high physiologic availability of 4-MC in the organism.

### TECHNICAL OBJECT OF THE INVENTION

It is therefore the technical object of the invention to specify means, which are suitable to prevent or treat the above diseases while ensuring a high physiologic availability of 4 MC or, if applicable, of the physiologically effective derivatives thereof.

Basics of the invention and preferred embodiments

For achieving this technical object, the invention teaches a compound of the general Formula I:

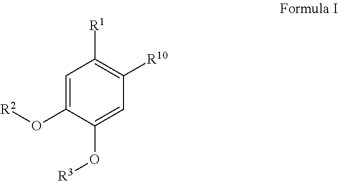

wherein R1 and R10, identical or different, is selected from —H, —OH, C1-C6 alkyl, linear or branched, saturated or not saturated, unsubstituted or substituted with —COOR4, —CONR5R6, and/or —NR5R6, wherein R4, R5 and R6, identical or different, is selected from —H, —C1-C6 alkyl, linear or branched, saturated or not saturated, or —(CH2)n-COOR7, with n=1-5, and R7 is —H or —C1-C6 alkyl, linear or branched, saturated or not saturated, wherein R2 and R3 are moieties separable microbiotically or by the human organism, identical or different, which when separated are physiologically tolerable, and wherein after separation of the moieties R2 and R3 a catechol derivative with R1 in position 4 is formed (i.e., with R2 and R3, after separation, being then replaced by —H), and wherein R1 alternatively may be arranged vicinally to the group —O—R2, not however a compound from the group consisting of 5-allyl-catechol-bis(β-D-glucopyranoside), 3-O-aα-D-glucopyranosyl-dopamine, 4-O-α-D-glucopyranosyl-dopamine, 3-O-α-D-glucopyranosyl-DOPA, 4-O-α-D-glucopyranosyl-DOPA, 3-O-α-D-manopyranosyl-dopamine, 4-O-α-D-manopyranosyl-dopamine, 3-O-α-D-galactopyranosyl-dopamine, 4-O-α-D-galactopyranosyl-dopamine, 3-O-α-D-2-desoxyglucopyranosyl-dopamine, 4-O-α-D-2-desoxyglucopyranosyl-dopamine, 3-O-α-D-O-methyl-glucopyranosyl-dopamine, 4-O-α-D-O-methylglucopyranosyl-dopamine, dihydro-caffeic acid-diglucoside, 3,4-dihydroxy-allylbenzene-3-O-α-L-rhamnopyranosyl (1→6)-β-D-glucopyranoside, and 3,4-dihydroxy-allylbenzene-3-O-α-L-rhamnopyranosyl (1→2)-β-D-gluco-pyranoside.

In the above disclaimed substances, it is the compound 21 of WO 2010/075282 A1, formula Ï of ES 2,156,777, compounds of FIG. 4 of Seung-Heon Yoon et al., Carbohydrate Research 334 (2009) 2349-2356, compound 12 of Table 4 of Helmja K. et al., Electrophoresis 2008. 29, 3980-3988, and compounds 1 and 2 of Deng, S. M. et al, Chinese Chemical Letters Vol. 11, No. 11, pp. 1001-1002, 2000.

Alternatively to the above disclaimers, it may be provided that R1 is not —CH₂—CH═CH₂ or —CH₂—CH₂—NH₂ or —CH₂—CH(NH₂)—COOH or —CH═CH—COOH, preferably when R10 is —H, or vice versa.

In an independent alternative variation of the invention, R2 is a microbiotically not separable moiety, which is directly coupled to the aromatic ring of Formula I (also without the —O— atom shown in Formula I) and R3 is —H, wherein R2 contains at least one ionizable functional group, or physiologically tolerable salts of such compounds.

In another independent alternative variation of the invention, the compound comprises the structure according to Formula II, wherein the moieties R21, R22, R23 and R24, identical or different, can be configured according to the moiety R1, and wherein n=0-20, preferably n=0-10, in particular n=0-5.

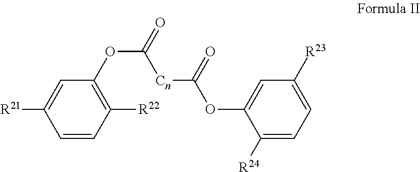

It is preferred, when R21 and R23 are C1-C6 alkyl, in particular methyl. It is further preferred, when R22 and R24 are —OH. Finally n=1-3 is preferred, in particular n=0 and n=1 and n=2.

Other than that, for both alternative variations apply in an analogous manner all explanations and uses etc. mentioned here in the context with compounds of Formula I. Physiologically tolerable salts comprise as counter ions for ionic compounds, for instance, Mg⁺⁺, Pb⁺⁺, Mn⁺⁺, Ca⁺⁺, CaCl⁺, Na⁺, K⁺, Li⁺ or cyclohexylammonium, or Cl⁻, Br⁻, acetate, trifluoroacetate, propionate, lactate, oxalate, malonate, maleinate, citrate, benzoate, salicylate, putrecin, cadaverin, spermidin, spermin, etc. are considered. Salts of the moieties excluded in claim 1 are however also excluded.

All these compounds are suitable to be transformed in the colon by the microbiota being active there into compounds, which have the physiologic activity of 4-MC. As will be explained further below, the galenic preparation may be carried out such that a metabolization in the stomach or in the small intestine will practically not occur.

Surprisingly, it has been shown that the microbiota of the large intestine plays a decisive role for a reproducible high availability of 4-MC for the human organism. With the present invention, a way is shown, how after oral administration compounds will arrive at the colon under protection from resorption in the stomach and small intestine, and here 4-MC is created from these compounds in an increased manner by the metabolic activity of the microbiota.

In the variation, where R2 is a moiety microbiotically not separable and directly bound to the ring C atom of Formula I, and R3 is —H, wherein R2 contains at least one ionizable functional group, this may in particular be a quercetin derivative, preferably quercetin sulfate, wherein the sulfate group is bound to a C atom carrying an —OH group of the quercetin molecule, or a glucoside, wherein the sugar moiety is glycosidically bound at the —O— atom of one of the —OH groups of the quercetin molecule. As sugars are considered the sugars mentioned in the following in other contexts. They may be present 1-fold, 2-fold, 3-fold, 4-fold, or 5-fold, identical or different. Further, the sulfate groups may be present 1-fold, 2-fold, 3-fold, 4-fold, or 5-fold. It is also possible to combine in an arbitrary way sugar group(s) and sulfate group(s). An example of a suitable compound is quercetin-3-sulfate. Further examples include all compounds and substances mentioned in the document DE patent application 10 2007 029 042.1, for instance:

2-(3,4-dihydroxyphenyl)-5,7-dihydroxy-4-oxo-4H-chromene-3-yl hydrogen sulfate;

5,7-dihydroxy-2-(3-hydroxy-4-methoxyphenyl)4-oxo-4H-chromene-3-yl hydrogen sulfate;

5,7-dihydroxy-2-(4-hydroxy-3-methoxyphenyl)4-oxo-4H-chromene-3-yl hydrogen sulfate;

2-(3,4-dimethoxyphenyl)-5,7-dihydroxy-4-oxo-4H-chromene-3-yl hydrogen sulfate;

2-hydroxy-5-(3,5,7-trihydroxy-4-oxo-4H-chromene-2-yl) phenyl hydrogen sulfate;

2-methoxy-5-(3,5,7-trihydroxy-4-oxo-4H-chromene-2-yl) phenyl hydrogen sulfate;

2-hydroxy-4-(3,5,7-trihydroxy-4-oxo-4H-chromene-2-yl) phenyl hydrogen sulfate;

2-methoxy-4-(3,5,7-trihydroxy-4-oxo-4H-chromene-2-yl) phenyl hydrogen sulfate;

2-(3,4-dihydroxyphenyl)-3,5-dihydroxy-4-oxo-4H-chromene-7-yl hydrogen sulfate;

2-(3,4-dimethoxyphenyl)-3,5-dihydroxy-4-oxo-4H-chromene-7-yl hydrogen sulfate;

3,5-dihydroxy-2-(4-hydroxy-3-methoxyphenyl)-4-oxo-4H-chromene-7-yl hydrogen sulfate;

3,5-dihydroxy-2-(3-hydroxy-4-methoxyphenyl)-4-oxo-4H-chromene-7-yl hydrogen sulfate;

6-{[2-(3,4-dihydroxyphenyl)-5,7-dihydroxy-4-oxo-4H-chromene-3-yl]oxy}-3,4,5-trihydroxytetrahydro-2H-pyran-2-carboxylic acid;

6-{[5,7-dihydroxy-2-(3-hydroxy-4-methoxyphenyl)4-oxo-4H-chromene-3-yl]oxy}-3,4,5-trihydroxytetrahydro-2H-pyran-2-carboxylic acid;

6-{[5,7-dihydroxy-2-(4-hydroxy-3-methoxyphenyl)4-oxo-4H-chromene-3-yl]oxy}-3,4,5-trihydroxytetrahydro-2H-pyran-2-carboxylic acid;

6-{[2-(3,4-dimethoxyphenyl)-5,7-dihydroxy-4-oxo-4H-chromene-3-yl]oxy}-3,4,5-trihydroxytetrahydro-2H-pyran-2-carboxylic acid;

6-{[2-(3,4-dihydroxyphenyl)-3,5-dihydroxy-4-oxo-4H-chromene-7-yl]oxy}-3,4,5-trihydroxytetrahydro-2-H-pyran-2-carboxylic acid;

6-{[2-(3,4-dimethoxyphenyl)-3,5-dihydroxy-4-oxo-4H-chromene-7-yl]oxy}-3,4,5-trihydroxytetrahydro-2-H-pyran-2-carboxylic acid;

6-{[2-(3-hydroxy-4-methoxyphenyl)-3,5-dihydroxy-4-oxo-4H-chromene-7-yl]oxy}-3,4,5-trihydroxytetrahydro-2-H-pyran-2-carboxylic acid;

6-{[2-(3-methoxy-4-hydroxyphenyl)-3,5-dihydroxy-4-oxo-4H-chromene-7-yl]oxy}-3,4,5-trihydroxytetrahydro-2-H-pyran-2-carboxylic acid;

3,5,7-trihydroxy-2-(3-hydroxy-4-{[3,4,5-trihydroxy-6-(hydroxymethyl)tetrahydro-2H-pyran-2-yl]oxy}phenyl)-4H-chromene-4-one;

3,5,7-trihydroxy-2-(4-hydroxy-3-{[3,4,5-trihydroxy-6-(hydroxymethyl)tetrahydro-2H-pyran-2-yl]oxy}phenyl)-4H-chromene-4-one;

5,7-dihydroxy-3-{[3,4,5-trihydroxy-6-(hydroxymethyl)tetrahydro-2H-pyran-2-yl]oxy}-2-(3,4-dihydroxyphenyl)-4H-chromene-4-one;

3,5,7-trihydroxy-2-(3-hydroxy-4-{[3,4,5-trihydroxy-6-(hydroxymethyl)tetrahydro-2H-pyran-2-yl]oxy}phenyl)-4H-chromene-4-one;

3,5-dihydroxy-7-{[3,4,5-trihydroxy-6-(hydroxymethyl)tetrahydro-2H-pyran-2-yl]oxy}-2-(3,4-dihydroxyphenyl)-4H-chromene-4-one,

and physiologically tolerable salts of these compounds.

It is preferred, when R1 is selected from —H, —OH, —CH₃, —CH₂—CH₃, —CH₂—COOH, or —CH₂—COO⁻.

It is further preferred, when R2 and R3, identical or different, are selected from —OR7, —O—CO—R8 and L-sugar moiety, in D or L form, as furanose, pyranose, each in alpha or beta form, or as an aldehyde, in particular L-sugar moieties of the L-rhamnose, L-lactulose, L-xylose, L-arabinose, L-mannose, L-glucose, and wherein R7 and R8 are selected from —H, —C1-C6 alkyl, linear or branched, saturated or not saturated. The term of the sugar moiety denotes the moiety of the sugar excluding the glycosidic —O— bridge. Typically, the glycosidic bond will be arranged in position 2 of the sugar molecule. It may however also be provided at one of the positions 3, 4, or 5 of the sugar molecule. Instead of the —O— bridge, there may however also be provided an —S— or —Se— bridge as a glycosidic bond, these may therefore be thioglycosides or selenoglycosides. This also applies for —OH groups, which may instead be —SH or —Se groups. These variations apply for the above general formulas, as well as for the special formulas or specific substances explained in the following.

It is possible that only one of the moieties R2 or R3 is an L-sugar moiety, and the other moiety is one of the mentioned not-glycosidic moieties (monoglycosides), or that both moieties R2 and R3 are an L-sugar moiety, identical or different (diglycosides).

In detail, it is preferred, when R2 is selected from one of the sugar moieties of the L-rhamnose, L-lactulose, L-xylose, L-arabinose, L-mannose, L-glucose, and R3 is —OH, or vice versa. Examples for compounds according to the invention, without being limiting, are stated in the following.

A glycoside of the L-rhamnose, wherein the aglycone moiety is 2-hydroxy-5-methylphenoxy,

a glycoside of the L-rhamnose, wherein the aglycone moiety is 2-hydroxy-5-ethylphenoxy,

a glycoside of the L-rhamnose, wherein the aglycone moiety is 5-(carboxymethyl)-2-hydroxyphenoxy,

a glycoside of the L-rhamnose, wherein the aglycone moiety is 5-(aminomethyl)-2-hydroxyphenoxy,

a glycoside of the L-rhamnose, wherein the aglycone moiety is 2-methoxy-5-methylphenoxy,

a glycoside of the L-rhamnose, wherein the aglycone moiety is 2-methoxy-5-ethylphenoxy,

a glycoside of the L-rhamnose, wherein the aglycone moiety is 5-(carboxymethyl)-2-methoxyphenoxy,

a glycoside of the L-rhamnose, wherein the aglycone moiety is 5-(aminomethyl)-2-methoxyphenoxy,

a glycoside of the L-rhamnose, wherein the aglycone moiety is 2-ethoxy-5-methylphenoxy,

a glycoside of the L-rhamnose, wherein the aglycone moiety is 2-ethoxy-5-ethylphenoxy,

a glycoside of the L-rhamnose, wherein the aglycone moiety is 5-(carboxymethyl)-2-ethoxyphenoxy,

a glycoside of the L-rhamnose, wherein the aglycone moiety is 5-(aminomethyl)-2-ethoxyphenoxy,

a glycoside of the L-rhamnose, wherein the aglycone moiety is 2-hydroxy-4-methylphenoxy,

a glycoside of the L-rhamnose, wherein the aglycone moiety is 2-hydroxy-4-ethylphenoxy,

a glycoside of the L-rhamnose, wherein the aglycone moiety is 5-(carboxymethyl)-2-hydroxyphenoxy,

a glycoside of the L-rhamnose, wherein the aglycone moiety is 5-(aminomethyl)-2-hydroxyphenoxy,

a glycoside of the L-rhamnose, wherein the aglycone moiety is 2-methoxy-4-methylphenoxy,

a glycoside of the L-rhamnose, wherein the aglycone moiety is 2-methoxy-4-ethylphenoxy,

a glycoside of the L-rhamnose, wherein the aglycone moiety is 4-(carboxymethyl)-2-methoxyphenoxy,

a glycoside of the L-rhamnose, wherein the aglycone moiety is 4-(aminomethyl)-2-methoxyphenoxy,

a glycoside of the L-rhamnose, wherein the aglycone moiety is 2-ethoxy-4-methylphenoxy,

a glycoside of the L-rhamnose, wherein the aglycone moiety is 2-ethoxy-4-ethylphenoxy,

a glycoside of the L-rhamnose, wherein the aglycone moiety is 4-(carboxymethyl)-2-ethoxyphenoxy,

a glycoside of the L-rhamnose, wherein the aglycone moiety is 4-(aminomethyl)-2-ethoxyphenoxy,

a glycoside of the L-lactulose, wherein the aglycone moiety is 2-hydroxy-5-methylphenoxy,

a glycoside of the L-lactulose, wherein the aglycone moiety is 2-hydroxy-5-ethylphenoxy,

a glycoside of the L-lactulose, wherein the aglycone moiety is 5-(carboxymethyl)-2-hydroxyphenoxy,

a glycoside of the L-lactulose, wherein the aglycone moiety is 5-(aminomethyl)-2-hydroxyphenoxy,

a glycoside of the L-lactulose, wherein the aglycone moiety is 2-methoxy-5-methylphenoxy,

a glycoside of the L-lactulose, wherein the aglycone moiety is 2-methoxy-5-ethylphenoxy,

a glycoside of the L-lactulose, wherein the aglycone moiety is 5-(carboxymethyl)-2-methoxyphenoxy,

a glycoside of the L-lactulose, wherein the aglycone moiety is 5-(aminomethyl)-2-methoxyphenoxy,

a glycoside of the L-lactulose, wherein the aglycone moiety is 2-ethoxy-5-methylphenoxy,

a glycoside of the L-lactulose, wherein the aglycone moiety is 2-ethoxy-5-ethylphenoxy,

a glycoside of the L-lactulose, wherein the aglycone moiety is 5-(carboxymethyl)-2-ethoxyphenoxy,

a glycoside of the L-lactulose, wherein the aglycone moiety is 5-(aminomethyl)-2-ethoxyphenoxy,

a glycoside of the L-lactulose, wherein the aglycone moiety is 2-hydroxy-4-methylphenoxy,

a glycoside of the L-lactulose, wherein the aglycone moiety is 2-hydroxy-4-ethylphenoxy,

a glycoside of the L-lactulose, wherein the aglycone moiety is 5-(carboxymethyl)-2-hydroxyphenoxy,

a glycoside of the L-lactulose, wherein the aglycone moiety is 5-(aminomethyl)-2-hydroxyphenoxy,

a glycoside of the L-lactulose, wherein the aglycone moiety is 2-methoxy-4-methylphenoxy,

a glycoside of the L-lactulose, wherein the aglycone moiety is 2-methoxy-4-ethylphenoxy,

a glycoside of the L-lactulose, wherein the aglycone moiety is 4-(carboxymethyl)-2-methoxyphenoxy,

a glycoside of the L-lactulose, wherein the aglycone moiety is 4-(aminomethyl)-2-methoxyphenoxy,

a glycoside of the L-lactulose, wherein the aglycone moiety is 2-ethoxy-4-methylphenoxy,

a glycoside of the L-lactulose, wherein the aglycone moiety is 2-ethoxy-4-ethylphenoxy,

a glycoside of the L-lactulose, wherein the aglycone moiety is 4-(carboxymethyl)-2-ethoxyphenoxy,

a glycoside of the L-lactulose, wherein the aglycone moiety is 4-(aminomethyl)-2-ethoxyphenoxy,

a glycoside of the L-xylose, wherein the aglycone moiety is 2-hydroxy-5-methylphenoxy,

a glycoside of the L-xylose, wherein the aglycone moiety is 2-hydroxy-5-ethylphenoxy,

a glycoside of the L-xylose, wherein the aglycone moiety is 5-(carboxymethyl)-2-hydroxyphenoxy,

a glycoside of the L-xylose, wherein the aglycone moiety is 5-(aminomethyl)-2-hydroxyphenoxy,

a glycoside of the L-xylose, wherein the aglycone moiety is 2-methoxy-5-methylphenoxy,

a glycoside of the L-xylose, wherein the aglycone moiety is 2-methoxy-5-ethylphenoxy,

a glycoside of the L-xylose, wherein the aglycone moiety is 5-(carboxymethyl)-2-methoxyphenoxy,

a glycoside of the L-xylose, wherein the aglycone moiety is 5-(aminomethyl)-2-methoxyphenoxy,

a glycoside of the L-xylose, wherein the aglycone moiety is 2-ethoxy-5-methylphenoxy,

a glycoside of the L-xylose, wherein the aglycone moiety is 2-ethoxy-5-ethylphenoxy,

a glycoside of the L-xylose, wherein the aglycone moiety is 5-(carboxymethyl)-2-ethoxyphenoxy,

a glycoside of the L-xylose, wherein the aglycone moiety is 5-(aminomethyl)-2-ethoxyphenoxy,

a glycoside of the L-xylose, wherein the aglycone moiety is 2-hydroxy-4-methylphenoxy,

a glycoside of the L-xylose, wherein the aglycone moiety is 2-hydroxy-4-ethylphenoxy,

a glycoside of the L-xylose, wherein the aglycone moiety is 5-(carboxymethyl)-2-hydroxyphenoxy,

a glycoside of the L-xylose, wherein the aglycone moiety is 5-(aminomethyl)-2-hydroxyphenoxy,

a glycoside of the L-xylose, wherein the aglycone moiety is 2-methoxy-4-methylphenoxy,

a glycoside of the L-xylose, wherein the aglycone moiety is 2-methoxy-4-ethylphenoxy,

a glycoside of the L-xylose, wherein the aglycone moiety is 4-(carboxymethyl)-2-methoxyphenoxy,

a glycoside of the L-xylose, wherein the aglycone moiety is 4-(aminomethyl)-2-methoxyphenoxy,

a glycoside of the L-xylose, wherein the aglycone moiety is 2-ethoxy-4-methylphenoxy,

a glycoside of the L-xylose, wherein the aglycone moiety is 2-ethoxy-4-ethylphenoxy,

a glycoside of the L-xylose, wherein the aglycone moiety is 4-(carboxymethyl)-2-ethoxyphenoxy,

a glycoside of the L-xylose, wherein the aglycone moiety is 4-(aminomethyl)-2-ethoxyphenoxy,

a glycoside of the L-arabinose, wherein the aglycone moiety is 2-hydroxy-5-methylphenoxy,

a glycoside of the L-arabinose, wherein the aglycone moiety is 2-hydroxy-5-ethylphenoxy,

a glycoside of the L-arabinose, wherein the aglycone moiety is 5-(carboxymethyl)-2-hydroxyphenoxy,

a glycoside of the L-arabinose, wherein the aglycone moiety is 5-(aminomethyl)-2-hydroxyphenoxy,

a glycoside of the L-arabinose, wherein the aglycone moiety is 2-methoxy-5-methylphenoxy,

a glycoside of the L-arabinose, wherein the aglycone moiety is 2-methoxy-5-ethylphenoxy,

a glycoside of the L-arabinose, wherein the aglycone moiety is 5-(carboxymethyl)-2-methoxyphenoxy,

a glycoside of the L-arabinose, wherein the aglycone moiety is 5-(aminomethyl)-2-methoxyphenoxy,

a glycoside of the L-arabinose, wherein the aglycone moiety is 2-ethoxy-5-methylphenoxy,

a glycoside of the L-arabinose, wherein the aglycone moiety is 2-ethoxy-5-ethylphenoxy,

a glycoside of the L-arabinose, wherein the aglycone moiety is 5-(carboxymethyl)-2-ethoxyphenoxy,

a glycoside of the L-arabinose, wherein the aglycone moiety is 5-(aminomethyl)-2-ethoxyphenoxy,

a glycoside of the L-arabinose, wherein the aglycone moiety is 2-hydroxy-4-methylphenoxy,

a glycoside of the L-arabinose, wherein the aglycone moiety is 2-hydroxy-4-ethylphenoxy,

a glycoside of the L-arabinose, wherein the aglycone moiety is 5-(carboxymethyl)-2-hydroxyphenoxy,

a glycoside of the L-arabinose, wherein the aglycone moiety is 5-(aminomethyl)-2-hydroxyphenoxy,

a glycoside of the L-arabinose, wherein the aglycone moiety is 2-methoxy-4-methylphenoxy,

a glycoside of the L-arabinose, wherein the aglycone moiety is 2-methoxy-4-ethylphenoxy,

a glycoside of the L-arabinose, wherein the aglycone moiety is 4-(carboxymethyl)-2-methoxyphenoxy,

a glycoside of the L-arabinose, wherein the aglycone moiety is 4-(aminomethyl)-2-methoxyphenoxy,

a glycoside of the L-arabinose, wherein the aglycone moiety is 2-ethoxy-4-methylphenoxy,

a glycoside of the L-arabinose, wherein the aglycone moiety is 2-ethoxy-4-ethylphenoxy,

a glycoside of the L-arabinose, wherein the aglycone moiety is 4-(carboxymethyl)-2-ethoxyphenoxy,

a glycoside of the L-arabinose, wherein the aglycone moiety is 4-(aminomethyl)-2-ethoxyphenoxy,

a glycoside of the L-mannose, wherein the aglycone moiety is 2-hydroxy-5-methylphenoxy,

a glycoside of the L-mannose, wherein the aglycone moiety is 2-hydroxy-5-ethylphenoxy,

a glycoside of the L-mannose, wherein the aglycone moiety is 5-(carboxymethyl)-2-hydroxyphenoxy,

a glycoside of the L-mannose, wherein the aglycone moiety is 5-(aminomethyl)-2-hydroxyphenoxy,

a glycoside of the L-mannose, wherein the aglycone moiety is 2-methoxy-5-methylphenoxy,

a glycoside of the L-mannose, wherein the aglycone moiety is 2-methoxy-5-ethylphenoxy,

a glycoside of the L-mannose, wherein the aglycone moiety is 5-(carboxymethyl)-2-methoxyphenoxy,

a glycoside of the L-mannose, wherein the aglycone moiety is 5-(aminomethyl)-2-methoxyphenoxy,

a glycoside of the L-mannose, wherein the aglycone moiety is 2-ethoxy-5-methylphenoxy,

a glycoside of the L-mannose, wherein the aglycone moiety is 2-ethoxy-5-ethylphenoxy,

a glycoside of the L-mannose, wherein the aglycone moiety is 5-(carboxymethyl)-2-ethoxyphenoxy,

a glycoside of the L-mannose, wherein the aglycone moiety is 5-(aminomethyl)-2-ethoxyphenoxy,

a glycoside of the L-mannose, wherein the aglycone moiety is 2-hydroxy-4-methylphenoxy,

a glycoside of the L-mannose, wherein the aglycone moiety is 2-hydroxy-4-ethylphenoxy,

a glycoside of the L-mannose, wherein the aglycone moiety is 5-(carboxymethyl)-2-hydroxyphenoxy,

a glycoside of the L-mannose, wherein the aglycone moiety is 5-(aminomethyl)-2-hydroxyphenoxy,

a glycoside of the L-mannose, wherein the aglycone moiety is 2-methoxy-4-methylphenoxy,

a glycoside of the L-mannose, wherein the aglycone moiety is 2-methoxy-4-ethylphenoxy,

a glycoside of the L-mannose, wherein the aglycone moiety is 4-(carboxymethyl)-2-methoxyphenoxy,

a glycoside of the L-mannose, wherein the aglycone moiety is 4-(aminomethyl)-2-methoxyphenoxy,

a glycoside of the L-mannose, wherein the aglycone moiety is 2-ethoxy-4-methylphenoxy,

a glycoside of the L-mannose, wherein the aglycone moiety is 2-ethoxy-4-ethylphenoxy,

a glycoside of the L-mannose, wherein the aglycone moiety is 4-(carboxymethyl)-2-ethoxyphenoxy,

a glycoside of the L-mannose, wherein the aglycone moiety is 4-(aminomethyl)-2-ethoxyphenoxy,

a glycoside of the L-glucose, wherein the aglycone moiety is 2-hydroxy-5-methylphenoxy,

a glycoside of the L-glucose, wherein the aglycone moiety is 2-hydroxy-5-ethylphenoxy,

a glycoside of the L-glucose, wherein the aglycone moiety is 5-(carboxymethyl)-2-hydroxyphenoxy,

a glycoside of the L-glucose, wherein the aglycone moiety is 5-(aminomethyl)-2-hydroxyphenoxy,

a glycoside of the L-glucose, wherein the aglycone moiety is 2-methoxy-5-methylphenoxy,

a glycoside of the L-glucose, wherein the aglycone moiety is 2-methoxy-5-ethylphenoxy,

a glycoside of the L-glucose, wherein the aglycone moiety is 5-(carboxymethyl)-2-methoxyphenoxy,

a glycoside of the L-glucose, wherein the aglycone moiety is 5-(aminomethyl)-2-methoxyphenoxy,

a glycoside of the L-glucose, wherein the aglycone moiety is 2-ethoxy-5-methylphenoxy,

a glycoside of the L-glucose, wherein the aglycone moiety is 2-ethoxy-5-ethylphenoxy,

a glycoside of the L-glucose, wherein the aglycone moiety is 5-(carboxymethyl)-2-ethoxyphenoxy,

a glycoside of the L-glucose, wherein the aglycone moiety is 5-(aminomethyl)-2-ethoxyphenoxy,

a glycoside of the L-glucose, wherein the aglycone moiety is 2-hydroxy-4-methylphenoxy,

a glycoside of the L-glucose, wherein the aglycone moiety is 2-hydroxy-4-ethylphenoxy,

a glycoside of the L-glucose, wherein the aglycone moiety is 5-(carboxymethyl)-2-hydroxyphenoxy,

a glycoside of the L-glucose, wherein the aglycone moiety is 5-(aminomethyl)-2-hydroxyphenoxy,

a glycoside of the L-glucose, wherein the aglycone moiety is 2-methoxy-4-methylphenoxy,

a glycoside of the L-glucose, wherein the aglycone moiety is 2-methoxy-4-ethylphenoxy,

a glycoside of the L-glucose, wherein the aglycone moiety is 4-(carboxymethyl)-2-methoxyphenoxy,

a glycoside of the L-glucose, wherein the aglycone moiety is 4-(aminomethyl)-2-methoxyphenoxy,

a glycoside of the L-glucose, wherein the aglycone moiety is 2-ethoxy-4-methylphenoxy,

a glycoside of the L-glucose, wherein the aglycone moiety is 2-ethoxy-4-ethylphenoxy,

a glycoside of the L-glucose, wherein the aglycone moiety is 4-(carboxymethyl)-2-ethoxyphenoxy,

a glycoside of the L-glucose, wherein the aglycone moiety is 4-(aminomethyl)-2-ethoxyphenoxy,

wherein each of the sugar moieties may be present in the aldehyde form, the furanose form, or the pyranose form.

The term of the aglycone moiety denotes the non-sugar moiety with the glycosidic —O— (or —S— or —Se—). This is bound, on the one hand, to the phenyl ring and, on the other hand, to one of the C atoms of the sugar with number 2, 3, or 4.

The sugar moieties may also be dimers or oligomers, for instance, with 3, 4, or 5 sugar monomers, wherein the sugar monomers may be identical or different and may be linked with each other via 1-4 or 1-6.

The invention further relates to a pharmaceutical composition containing a compound according to the invention and to galenic excipients and/or carriers, prepared for oral administration, wherein the oral preparation preferably comprises a coat of the compound with a film or a capsule, the material of which is stable under the biological conditions in the stomach and in the small intestine. The term “stable” denotes that less than 50% by weight, in particular less than 20% by weight, preferably less than 5% by weight, of the introduced compound are degraded in the stomach or the small intestine, or that the complement of these values to 100% by weight of the compound arrives in the large intestine.

Such materials are well known to the person skilled in the art. As examples only are cited: anionic copolymers based on methacrylic acid and methyl-methacrylate, such as eudragit, in particular eudragit S, galactomannan, in particular ethylated guaran (guar galactomannan), dextran and poly-galactomannan fatty acid esters, in particular esters with lauric acid, amylose, in particular crosslinked amylose, chitosan, crosslinked chondroitin, pectin (Bauer Kh., Colonic drug delivery: review of material trends, American Pharma Review 2001, 4, 8-16).

A pharmaceutical composition according to the invention contains the compound according to the invention in a physiologically effective dosage. Such a dosage of an administered unit is typically, but not necessarily, in the range from 0.1 mg to 2,000 mg, preferably in the range from 1 mg to 500 mg, in particular in the range from 10 mg to 200 mg.

The invention also relates to a method of making a pharmaceutical composition according to the invention, wherein a compound according to the invention is mixed in a physiologically effective dosage with galenic excipients and carriers and is prepared to a predetermined form of administration.

Suitable solid or liquid galenic forms of preparation are, for instance, granules, powders, dragées, tablets, (micro-) capsules, suppositories, syrups, juices, suspensions, emulsions, and preparations with controlled release of the active substance, for the production of which usual excipients such as carriers, disintegrants, binders, coatings, swelling agents, gliding agents or lubricants, flavoring agents, sweeteners, and dissolution promoters, are used. It is further possible to encapsulate the active substance in nanocapsules being degradable preferably biologically, for instance, in the colon, but not in the stomach or the small intestine, or to introduce it into the pores of porous nanoparticles, biologically degradable, for instance, in the colon, but not in the stomach or in the small intestine, or stably. Excipients include, for instance, sodium carbonate, magnesium carbonate, magnesium bicarbonate, titanium dioxide, lactose, mannite and other sugars, talcum, milk protein, gelatin, starch, cellulose and their derivatives, animal and vegetable oils such as codliver, sunflower, peanut or sesame oil, polyethylene glycols and solvents, such as sterile water and mono- or multivalent alcohols, for instance, glycerol. A pharmaceutical composition according to the invention can be produced by that at least one substance used according to the invention is mixed in a defined dosage with a pharmaceutically suitable and physiologically tolerable carrier and, if applicable, further suitable active, additional or auxiliary substances with a defined dosage and prepared to the desired form of administration. Dilution agents include polyglycols, water, and buffer solutions. Suitable buffer substances are, for instance, N,N′-dibenzylethylenediamine, diethanolamine, ethylenediamine, N-methylglucamine, N-benzylphenethylamine, diethylamine, phosphate, sodium bicarbonate, or sodium carbonate. It however also possible to omit the dilution agent. Physiologically tolerable salts are salts with inorganic or organic acids, such as lactic acid, hydrochloric acid, sulfuric acid, acetic acid, citric acid, p-toluenesulfonic acid, or with inorganic or organic bases, such as NaOH, KOH, Mg(OH)₂, diethanolamine, ethylenediamine, or with amino acids, such as arginine, lysine, glutamic acid etc. or with inorganic salts, such as CaCl₂, NaCl or the free ions thereof, such as Ca²⁺, Na⁺, Pb⁺⁺, Cl⁻, SO₄²⁻ or corresponding salts and free ions of Mg⁺⁺ or Mn⁺⁺, or combinations thereof. They are produced to standard methods.

Furthermore, the invention relates to the use of a compound according to the invention for making a pharmaceutical composition, in particular for the prophylaxis or treatment of a disease of man or animal from the group consisting of peripheral and autonomic neuropathies, central nervous degenerative diseases, high blood pressure, arteriosclerosis, venous insufficiency, diabetes mellitus, osteoporosis, cataract, and photoaging of the skin.

Finally, the invention relates to a method for the prophylaxis or treatment of a disease of man or animal from the group consisting of peripheral and autonomic neuropathies, central nervous degenerative diseases, high blood pressure, arteriosclerosis, venous insufficiency, diabetes mellitus, osteoporosis, cataract, and photoaging of the skin, wherein to an organism, which falls ill or is at risk of falling ill with the disease, a compound according to the invention or a pharmaceutical composition according to the invention is administered in a predetermined physiologically effective dosage. Suitable daily dosages are, for instance, 0.3 mg to 6,000 mg, preferably 1 mg to 1,000 mg, in particular 10 mg to 500 mg.

Finally, the invention relates to a method of making a compound according to the invention, wherein a sugar, monomer, dimer, or oligomer is reacted with a protective group compound, wherein OH groups of the sugar are protected, wherein optionally an OH group of the sugar remains without a protective group, wherein the sugar is then reacted with a compound of Formula III

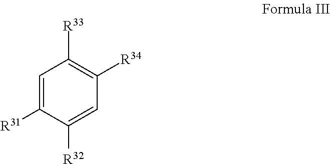

wherein R31 and R32, identical or different, are selected from —OH and —O—R35 with R35 being —H or —C1-C6 alkyl, linear or branched, saturated or not saturated, with the proviso that at least one of the moieties R31 or R32 is —OH, wherein R33 and R34, identical or different, may have the same meaning as R1 or is —CHO,

wherein the product of this reaction is freed from protective groups, wherein protected OH groups are reacted again to free OH groups, and

wherein before or after freeing the protective groups, the product is optionally derivatized in the area of the moieties R33 and/or R34 and/or, in case that one of the moieties R31 or R32 in the product is —OH, R31 or R32.

R31 and/or R32 and/or R34 may in particular be —H or —CH₃. R33 may in particular be R1.

Examples for protective groups, reagents therefor, reaction conditions, and the separation of protective groups and the reaction conditions thereof can be found in embodiments, irrespective of their actual implementation. Derivatizations can be made in a conventional way.

### EXAMPLE 1

### Preparation of Compounds according to the Invention

### EXAMPLE 1.1

### Preparation of 2-hydroxy-5-methylphenyl-β-D-rhamnopyranoside or 2-(2-methoxy-5-methylphenoxy)tetrahydro-2H-pyran-3,4,5-triol

To a solution of 1,2,3,4-tetra-O-acetyl-α/β-D-rhamnopyranose (45.9 g, 0.13 mol, preparation, for instance, according to Journal of Medicinal Chemistry 1987, 30(8), 1521-1525, or Bioscience, Biotechnology and Biochemistry 1996, 60(12), 2038-2042), and 4-methylcatechol (32.4 g, 0.26 mol) in absolute dichloromethane (250 ml) is added dropwise within 30 min. at room temperature a 0.1 M boron trifluoride diethyl etherate solution (12.2 ml), and the reaction mixture is stirred for approx. 2 hrs at room temperature (DC control). For work up, the reaction solution is extracted with semi-concentrated aqueous NaHCO₃ solution (1×300 ml) and thereafter with semi-concentrated NaCl solution (200 ml). The organic phase is dried over Na₂SO₄, filtered and concentrated in vacuum. Further purification of the raw product occurs by crystallization from acetic acid ethyl ester and subsequent slurrying/stirring with diisopropyl ether (100 ml). After another recrystallization from MeOH (100 ml), the compound 2-hydroxy-5-methylphenyl-2,3,4-tri-O-acetyl-β-D-rhamnopyranoside is obtained as a colorless solid (5.8 g, 11.3%). LC/MS: calc.: C₁₉H₂₄O₉ (396.4), found: [M+Na⁺] 419.5.

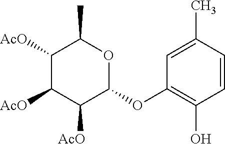 [SMILES: CC(=O)O[C@@H]1[C@H](OC(C)=O)[C@@H](Oc2cc(C)ccc2O)O[C@H](C)[C@H]1OC(C)=O]

To a solution of correspondingly synthesized 2-hydroxy-5-methylphenyl-2,3,4-tri-O-acetyl-β-D-rhamnopyranoside (5.75 g, 15.0 mmol) in absolute methanol (50 ml) is added under stirring at room temperature sodium methoxide (30%, 0.13 g, 1 mmol). After approx. 1 hr (DC control), the reaction solution is neutralized (pH=7) by adding an ion exchanger (Amberlite IR 120, H⁺ form). The ion exchanger is filtered off and re-washed with methanol (15 ml). The collected filtrates are concentrated in vacuum. The obtained raw product (3.2 g) is received in acetic acid ethyl ester (15 ml) and stirred. After filtration, the product according to the invention is obtained as a yellowish solid (4.0 g, 99%). LC/MS: calc.: C₁₃H₁₈O₇ (270.3), found: [M+Na⁺] 293, 230.

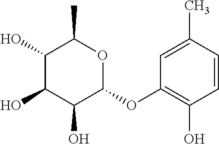 [SMILES: Cc1ccc(O)c(O[C@H]2O[C@H](C)[C@@H](O)[C@H](O)[C@@H]2O)c1]

### EXAMPLE 1.2

### Preparation of 2-hydroxy-5-methylphenyl-β-D-glucopyranoside or 2-(hydroxymethyl)-6-(2-methoxy-5-methylphenoxy)tetrahydro-2H-pyran-3.4.5-triol

To a solution of 1,2,3,4,6-penta-O-acetyl-α/β-D-glucopyranose (110.4 g, 0.28 mol, preparation, for instance, according to Journal of the American Chemical Society 1999, 121(51), 12196-12197, or Journal of Carbohydrate Chemistry 1997, 16(3), 327-342) and 4-methylcatechol (50.7 g, 0.40 mol) in absolute dichloromethane (500 ml) is added dropwise within 30 min. at room temperature a 0.1 M boron trifluoride diethyl etherate solution (85 ml) and the reaction mixture is stirred for approx. 2 hrs at room temperature (DC control). Thereafter, a 0.1 M boron trifluoride diethyl etherate solution (21 ml) is added dropwise to the reaction solution and is stirred for 1 hr at room temperature (DC control). For work up, the reaction solution is extracted with 1 M aqueous NaOH (1×500 ml). The organic phase is dried over Na₂SO₄, filtered and concentrated in vacuum. Further purification of the raw product occurs by flash column chromatography [heptane→heptane/EE (8:2)] and provides the title compound first as a yellow oil. After several crystallizations from hot EtOH and MeOH and subsequent slurrying/stirring with diisopropyl ether (200 ml), the compound 2-hydroxy-5-methylphenyl-2,3,4,6-tetra-O-acetyl-β-D-glucopyranoside is isolated as a colorless solid (22.8 g, 17.3%). LC/MS: calc.: O₂₁H₂₆O₁₁ (454.4), found: [M+Na⁺] 477.5.

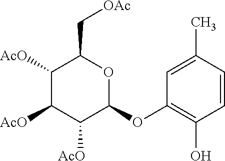 [SMILES: CC(=O)OC[C@H]1O[C@@H](Oc2cc(C)ccc2O)[C@H](OC(C)=O)[C@@H](OC(C)=O)[C@@H]1OC(C)=O]

To a solution of thus obtained 2-hydroxy-5-methylphenyl-2,3,4,6-tetra-O-acetyl-β-D-glucopyranoside (5.0 g, 11.0 mmol) in absolute methanol (50 ml) is added under stirring at room temperature sodium methoxide (30%, 0.2 g, 1 mmol). After approx. 4 hrs (DC control), the reaction solution is neutralized (pH=7) by adding an ion exchanger (Amberlite IR 120, H⁺ form). The ion exchanger is filtered off and re-washed with methanol (15 ml). The collected filtrates are concentrated in vacuum. The obtained raw product (3.2 g) is received in acetic acid ethyl ester (20 ml) and stirred. After filtration, the compound 2-hydroxy-5-methylphenyl-β-D-glucopyranoside is obtained as a colorless foam (3.0 g, 92%). LC/MS: calc.: C₁₃H₁₈O₇ (286.3), found: [M+Na⁺] 309.232.

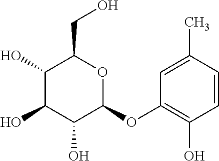 [SMILES: Cc1ccc(O)c(O[C@@H]2O[C@H](CO)[C@@H](O)[C@H](O)[C@H]2O)c1]

### EXAMPLE 1.3

### Preparation of 4-methylbrenzcatechin-bis (β-D-glucopyranoside)

To a solution of 2-hydroxy-5-methylphenyl-2,3,4,6-tetra-acetyl-β-D-glucopyranoside (10.1 g, 22.2 mmol (for preparation see Example 1.2) and (2,3,4,6-tetra-O-acetyl-α/β-D-glucopyranosyl)-trichloroacetimidate (16.3 g, 33.0 mmol, obtainable, for instance, according to the documents Liebigs Ann. Chem. 1984, 7, 1343-1357, Carb. Res. 2006, 342(12), 2115-2125, or Angew. Chem. Int. Ed. 2008, 47(18), 3396-3399) in absolute dichloromethane (100 ml), is added dropwise under ice cooling a 0.1 M boron trifluoride diethyl etherate solution (0.6 ml), and the reaction mixture is stirred for 2 hrs at 0° C. (DC control). For work up, the reaction solution is neutralized with triethylamine, diluted with acetic acid ethyl ester (100 ml) and extracted with water (200 ml). The organic phase is dried over Na₂SO₄, filtered off and concentrated in vacuum. Further purification occurs by flash column chromatography [heptane/EE (4:1)→heptane/EE (3:7)] and provides the compound 4-methylbrenzcatechin-bis(2,3,4,6-tetra-O-acetyl-β-D-glucopyranoside) (12.3 g, 71%) as a colorless foam. LC/MS: calc.: C₃₅H₄₄O₂₀ (784.7), found: [M+Na⁺] 807.6

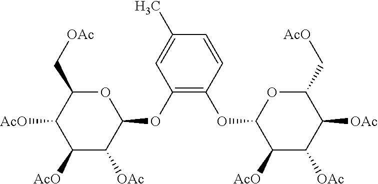 [SMILES: CC(=O)OC[C@H]1O[C@@H](Oc2ccc(C)cc2O[C@@H]2O[C@H](COC(C)=O)[C@@H](OC(C)=O)[C@H](OC(C)=O)[C@H]2OC(C)=O)[C@H](OC(C)=O)[C@@H](OC(C)=O)[C@@H]1OC(C)=O]

To a solution of 4-methylbrenzcatechin-bis-(2,3,4,6-tetra-O-acetyl-β-D-glucopyranoside) (6.0 g, 8.0 mmol) in absolute methanol (60 ml) is added under stirring at room temperature sodium methoxide (30%, 0.14 g, 1 mmol). After approx. 2 hrs (DC control), the reaction solution is neutralized (pH=7) by adding ion exchanger (Amberlite IR 120, H⁺ form). The ion exchanger is filtered off and re-washed with methanol (20 ml). The collected filtrates are concentrated in vacuum. The obtained raw product (3.6 g) is received in acetic acid ethyl ester (20 ml) and stirred. After filtration, the compound 4-methyl catechol-bis(β-D-glucopyranoside) is obtained as a lightly beige solid (3.2 g, 89%). LC/MS: calc.: C₁₉H₂₈O₁₂ (448.4), found: [M+Na⁺] 471.3.

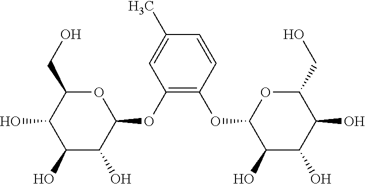 [SMILES: Cc1ccc(O[C@@H]2O[C@H](CO)[C@@H](O)[C@H](O)[C@H]2O)c(O[C@@H]2O[C@H](CO)[C@@H](O)[C@H](O)[C@H]2O)c1]

### EXAMPLE 1.4

### Further Substances according to the Invention

In the following, synthesis schemes for further substances according to the invention are graphically depicted. The educts, reagents, and reaction conditions correspond in an analogous manner to the examples 1.1 to 1.3.

1.4.1:

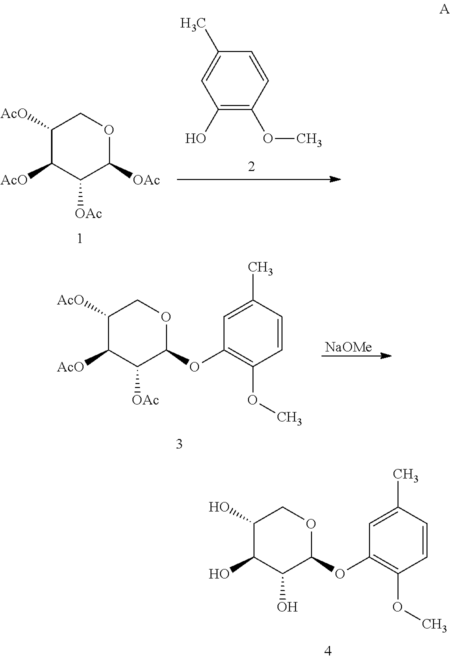

1.4.2:

1.

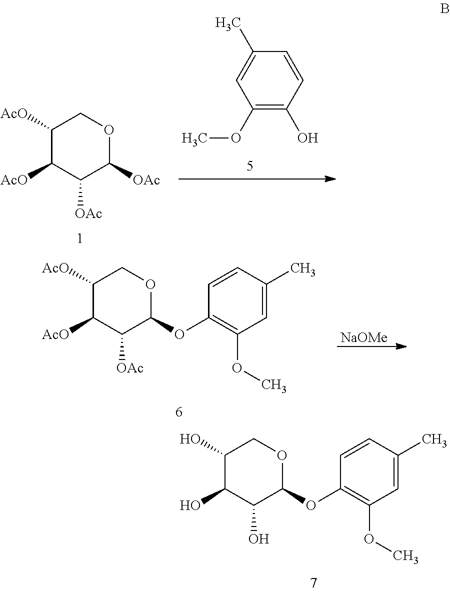

1.4.3:

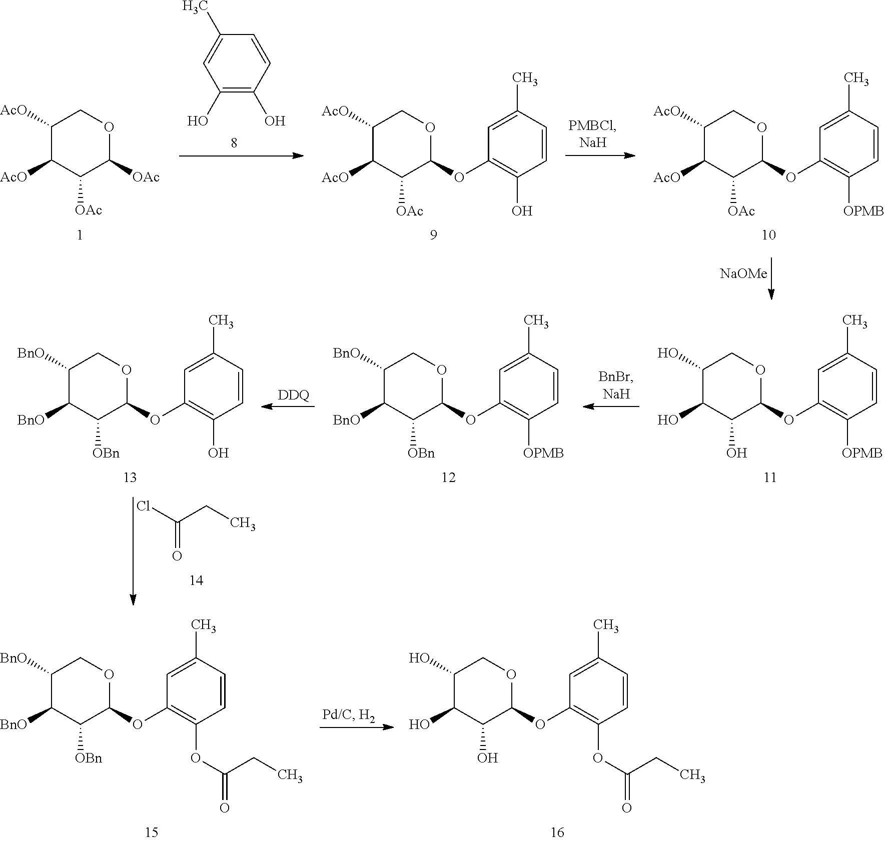

1.4.4:

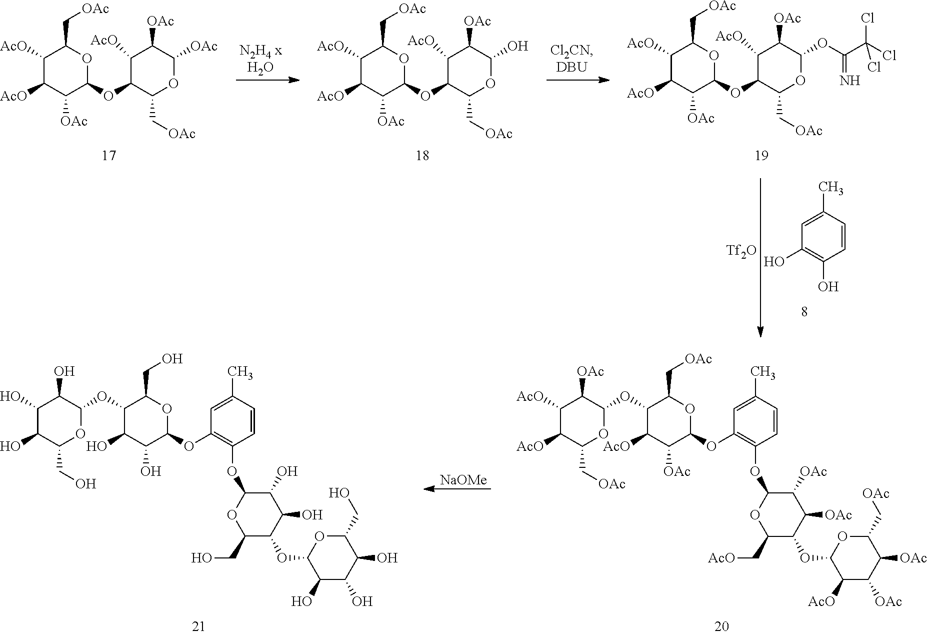

1.4.5:

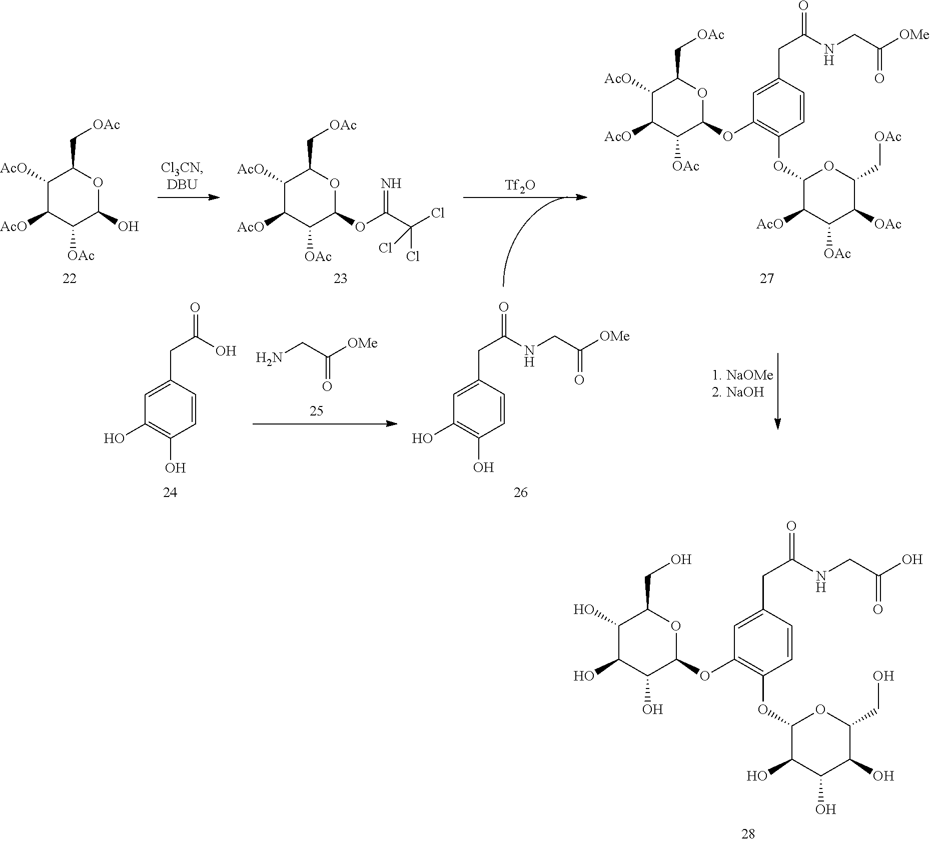

1.4.6:

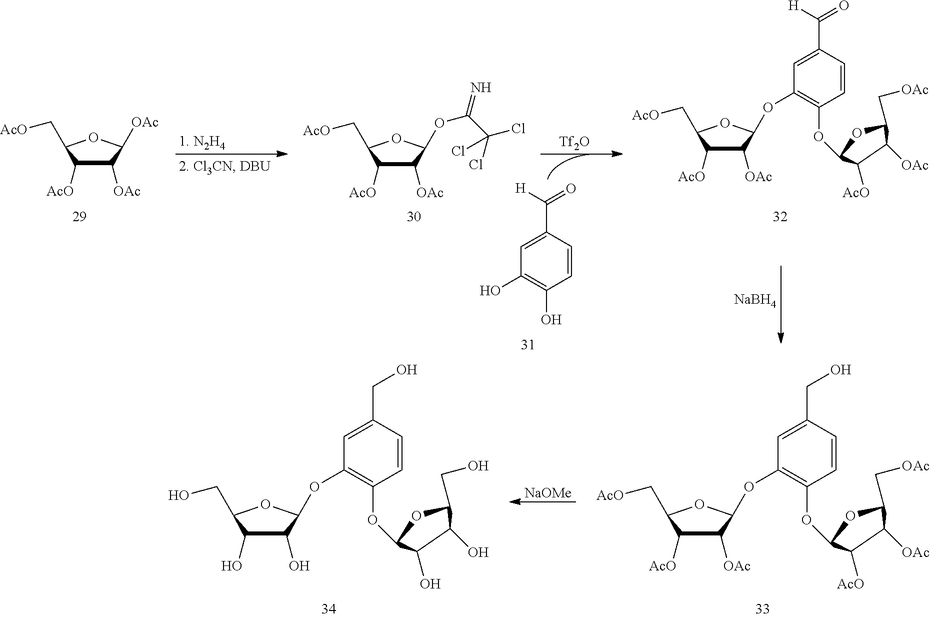

1.4.7:

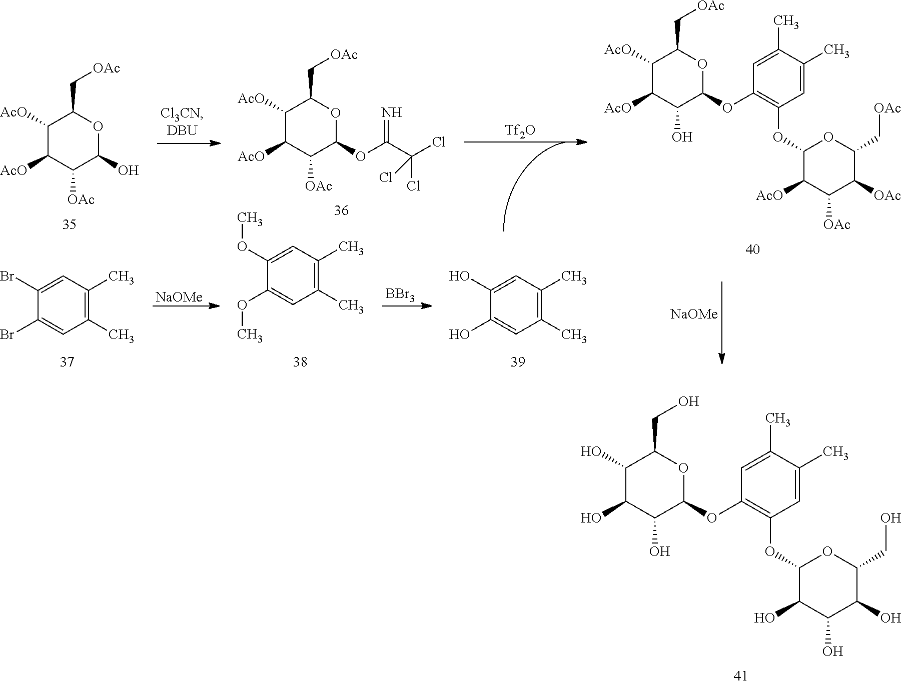

### EXAMPLE 2

### Galenic Preparation

In this example, the substance used according to the invention was alternatively prepared without a coat and with such a coat, and was employed for comparative experiments.

As a coat, eudragit S 12,5 (anionic copolymers based on methacrylic acid and methyl-methacrylate in the proportion 1:2 in isopropyl alcohol, obtainable from Evonik Industries) was used.

The form of administration was prepared by pressing together tablet cores with different amounts of the active substances (20 mg to 250 mg) and the auxiliary substances 6 mg magnesium stearate and 600 mg Ludipress (93% lactose plus 3.5% Kollidon plus Kollidon CL, obtainable from BASF).

Part of the tablet cores was coated with a 4% or 6% coat with eudragit S from an isopropanolic solution. Another part of the tablet cores did not receive any coat.

It can be seen that the product 4-MC generated in the metabolism and the O-methylated metabolites thereof, methylguaiacol and guaiacol—with a galenic preparation according to the invention of substances according to the invention—is discharged in the urine in a considerably higher amount and at later times (>6 h) after administration, compared to the administration without coat or capsule, which provides evidence that a considerably better physiologic availability is achieved with the invention.

TABLE 1
Excretion after administration of 50 mg
4-MC rhamnoside

| | |
|---|---|
| Tablets without | Tablets with |
| coat | eudragit coat |

4-MC total excret. [mg]

| | | |
|---|---|---|
| Person 1 | 5.0 | 13.5 |
| Person 2 | 4.8 | 10.3 |

4-MC excret. 0-12 h [% of total excret.]

| | | |
|---|---|---|
| Person 1 | 48.8 | 3.2 |
| Person 2 | 45.2 | 0.5 |

4-MC excret. 12-48 h [% of total excret.]

| | | |
|---|---|---|
| Person 1 | 51.2 | 96.8 |
| Person 2 | 54.8 | 99.3 |

4-methylguaiacol total excret. [mg]

| | | |
|---|---|---|
| Person 1 | 0.69 | 1.45 |
| Person 2 | 0.37 | 1.32 |

Guaiacol total excret.

| | | |
|---|---|---|
| Person 1 | 7.4 | 9.5 |
| Person 2 | 1.6 | 2.8 |

1. A compound of the general Formula I 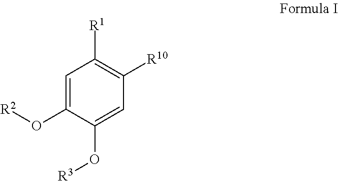
wherein R1 and R10, identical or different, is selected from —H, —OH, C1-C6 alkyl, linear or branched, saturated or not saturated, unsubstituted or substituted with —COOR4, —CONR5R6, and/or —NR5R6, wherein R4, R5 and R6, identical or different, is selected from —H, —C1-C6 alkyl, linear or branched, saturated or not saturated, or —(CH2)n-COOR7, with n=1-5, and R7 is —H or —C1-C6 alkyl, linear or branched, saturated or not saturated,
wherein R2 and R3 are moieties separable microbiotically or by the human organism, identical or different, which when separated are physiologically tolerable, and wherein after separation of the moieties R2 and R3 a catechol derivative with R1 in position 4 is formed, and wherein R1 alternatively may be arranged vicinally to the group —O—R2, or physiologically tolerable salts of such compounds, not however a compound from the group consisting of 5-allyl-catechol-bis(β-D-glucopyranoside), 3-O-α-D-glucopyranosyl-dopamine, 4-O-α-D-glucopyranosyl-dopamine, 3-O-α-D-glucopyranosyl-DOPA, 4-O-α-D-glucopyranosyl-DOPA, 3-O-α-D-manopyranosyl-dopamine, 4-O-α-D-manopyranosyl-dopamine, 3-O-α-D-galactopyranosyl-dopamine, 4-O-α-D-galactopyranosyl-dopamine, 3-O-α-D-2-desoxyglucopyranosyl-dopamine, 4-0-α-D-2-desoxyglucopyranosyl-dopamine, 3-O-α-D-O-methylglucopyranosyl-dopamine, 4-O-α-D-O-methylglucopyranosyl-dopamine, dihydro-caffeic acid-diglucoside, 3,4-dihydroxy-allylbenzene-3-O-α-L-rhamnopyranosyl (1→6)-β-D-glucopyranoside, and 3,4-dihydroxy-allylbenzene-3-O-α-L-rhamnopyranosyl(1→2)-β-D-glucopyranoside,
for the prophylaxis or treatment of a disease of man or animal from the group consisting of peripheral and autonomic neuropathies, central nervous degenerative diseases, high blood pressure, arteriosclerosis, venous insufficiency, diabetes mellitus, osteoporosis, cataract, and photoaging of the skin, or for the use of such a compound for the prophylaxis or treatment of such a disease.

2. The compound of claim 1, wherein R1 is selected from —H, —OH, —CH₃, —CH₂—CH₃, —CH₂—COOH, or —CH₂—COO⁻.

3. The compound of claim 1, wherein R2 and R3, identical or different, are selected from —H, —C1-C6 alkyl, linear or branched, saturated or not saturated, —CO—R8 and sugar moiety, in D or L form, as furanose, pyranose, each in alpha or beta form, or as an aldehyde, wherein the sugar moiety may be a monomer or oligomer with 2, 3, 4 or 5-10 sugar monomers, wherein the sugar monomers may be identical or different, in particular L-sugar moieties, monomer or hetero or homodimer, of the L-rhamnose, L-lactulose, L-xylose, L-arabinose, L-mannose, L-glucose, and wherein R8 is selected from —H, —C1-C6 alkyl, linear or branched, saturated or not saturated, wherein a glycosidic bond may be replaced by a thioglycosidic bond or a selenoglycosidic bond.

4. The compound of claim 3, wherein one of the moieties R2 or R3 is an L-sugar moiety, or wherein both moieties R2 and R3 are respectively an L-sugar moiety, identical or different.

5. The compound of claim 3, wherein R2 is selected from one of the sugar moieties of the L-rhamnose, L-lactulose, L-xylose, L-arabinose, L-mannose, L-glucose, and R3 is -H, or vice versa.

6. The compound of claim 1, selected from:
a glycoside of the L-rhamnose, wherein the aglycone moiety is 2-hydroxy-5-methylphenoxy,
a glycoside of the L-rhamnose, wherein the aglycone moiety is 2-hydroxy-5-ethylphenoxy,
a glycoside of the L-rhamnose, wherein the aglycone moiety is 5-(carboxymethyl)-2-hydroxyphenoxy,
a glycoside of the L-rhamnose, wherein the aglycone moiety is 5-(aminomethyl)-2-hydroxyphenoxy,
a glycoside of the L-rhamnose, wherein the aglycone moiety is 2-methoxy-5-methylphenoxy,
a glycoside of the L-rhamnose, wherein the aglycone moiety is 2-methoxy-5-ethylphenoxy,
a glycoside of the L-rhamnose, wherein the aglycone moiety is 5-(carboxymethyl)-2-methoxyphenoxy,
a glycoside of the L-rhamnose, wherein the aglycone moiety is 5-(aminomethyl)-2-methoxyphenoxy,
a glycoside of the L-rhamnose, wherein the aglycone moiety is 2-ethoxy-5-methylphenoxy,
a glycoside of the L-rhamnose, wherein the aglycone moiety is 2-ethoxy-5-ethylphenoxy,
a glycoside of the L-rhamnose, wherein the aglycone moiety is 5-(carboxymethyl)-2-ethoxyphenoxy,
a glycoside of the L-rhamnose, wherein the aglycone moiety is 5-(aminomethyl)-2-ethoxyphenoxy,
a glycoside of the L-rhamnose, wherein the aglycone moiety is 2-hydroxy-4-methylphenoxy,
a glycoside of the L-rhamnose, wherein the aglycone moiety is 2-hydroxy-4-ethylphenoxy,
a glycoside of the L-rhamnose, wherein the aglycone moiety is 5-(carboxymethyl)-2-hydroxyphenoxy,
a glycoside of the L-rhamnose, wherein the aglycone moiety is 5-(aminomethyl)-2-hydroxyphenoxy,
a glycoside of the L-rhamnose, wherein the aglycone moiety is 2-methoxy-4-methylphenoxy,
a glycoside of the L-rhamnose, wherein the aglycone moiety is 2-methoxy-4-ethylphenoxy,
a glycoside of the L-rhamnose, wherein the aglycone moiety is 4-(carboxymethyl)-2-methoxyphenoxy,
a glycoside of the L-rhamnose, wherein the aglycone moiety is 4-(aminomethyl)-2-methoxyphenoxy,
a glycoside of the L-rhamnose, wherein the aglycone moiety is 2-ethoxy-4-methylphenoxy,
a glycoside of the L-rhamnose, wherein the aglycone moiety is 2-ethoxy-4-ethylphenoxy,
a glycoside of the L-rhamnose, wherein the aglycone moiety is 4-(carboxymethyl)-2-ethoxyphenoxy,
a glycoside of the L-rhamnose, wherein the aglycone moiety is 4-(aminomethyl)-2-ethoxyphenoxy,
a glycoside of the L-lactulose, wherein the aglycone moiety is 2-hydroxy-5-methylphenoxy,
a glycoside of the L-lactulose, wherein the aglycone moiety is 2-hydroxy-5-ethylphenoxy,
a glycoside of the L-lactulose, wherein the aglycone moiety is 5-(carboxymethyl)-2-hydroxyphenoxy,
a glycoside of the L-lactulose, wherein the aglycone moiety is 5-(aminomethyl)-2-hydroxyphenoxy,
a glycoside of the L-lactulose, wherein the aglycone moiety is 2-methoxy-5-methylphenoxy,
a glycoside of the L-lactulose, wherein the aglycone moiety is 2-methoxy-5-ethylphenoxy,
a glycoside of the L-lactulose, wherein the aglycone moiety is 5-(carboxymethyl)-2-methoxyphenoxy,
a glycoside of the L-lactulose, wherein the aglycone moiety is 5-(aminomethyl)-2-methoxyphenoxy,
a glycoside of the L-lactulose, wherein the aglycone moiety is 2-ethoxy-5-methylphenoxy,
a glycoside of the L-lactulose, wherein the aglycone moiety is 2-ethoxy-5-ethylphenoxy,
a glycoside of the L-lactulose, wherein the aglycone moiety is 5-(carboxymethyl)-2-ethoxyphenoxy,
a glycoside of the L-lactulose, wherein the aglycone moiety is 5-(aminomethyl)-2-ethoxyphenoxy,
a glycoside of the L-lactulose, wherein the aglycone moiety is 2-hydroxy-4-methylphenoxy,
a glycoside of the L-lactulose, wherein the aglycone moiety is 2-hydroxy-4-ethylphenoxy,
a glycoside of the L-lactulose, wherein the aglycone moiety is 5-(carboxymethyl)-2-hydroxyphenoxy,
a glycoside of the L-lactulose, wherein the aglycone moiety is 5-(aminomethyl)-2-hydroxyphenoxy,
a glycoside of the L-lactulose, wherein the aglycone moiety is 2-methoxy-4-methylphenoxy,
a glycoside of the L-lactulose, wherein the aglycone moiety is 2-methoxy-4-ethylphenoxy,
a glycoside of the L-lactulose, wherein the aglycone moiety is 4-(carboxymethyl)-2-methoxyphenoxy,
a glycoside of the L-lactulose, wherein the aglycone moiety is 4-(aminomethyl)-2-methoxyphenoxy,
a glycoside of the L-lactulose, wherein the aglycone moiety is 2-ethoxy-4-methylphenoxy,
a glycoside of the L-lactulose, wherein the aglycone moiety is 2-ethoxy-4-ethylphenoxy,
a glycoside of the L-lactulose, wherein the aglycone moiety is 4-(carboxymethyl)-2-ethoxyphenoxy,
a glycoside of the L-lactulose, wherein the aglycone moiety is 4-(aminomethyl)-2-ethoxyphenoxy,
a glycoside of the L-xylose, wherein the aglycone moiety is 2-hydroxy-5-methylphenoxy,
a glycoside of the L-xylose, wherein the aglycone moiety is 2-hydroxy-5-ethylphenoxy,
a glycoside of the L-xylose, wherein the aglycone moiety is 5-(carboxymethyl)-2-hydroxyphenoxy,
a glycoside of the L-xylose, wherein the aglycone moiety is 5-(aminomethyl)-2-hydroxyphenoxy,
a glycoside of the L-xylose, wherein the aglycone moiety is 2-methoxy-5-methylphenoxy,
a glycoside of the L-xylose, wherein the aglycone moiety is 2-methoxy-5-ethylphenoxy,
a glycoside of the L-xylose, wherein the aglycone moiety is 5-(carboxymethyl)-2-methoxyphenoxy,
a glycoside of the L-xylose, wherein the aglycone moiety is 5-(aminomethyl)-2-methoxyphenoxy,
a glycoside of the L-xylose, wherein the aglycone moiety is 2-ethoxy-5-methylphenoxy,
a glycoside of the L-xylose, wherein the aglycone moiety is 2-ethoxy-5-ethylphenoxy,
a glycoside of the L-xylose, wherein the aglycone moiety is 5-(carboxymethyl)-2-ethoxyphenoxy,
a glycoside of the L-xylose, wherein the aglycone moiety is 5-(aminomethyl)-2-ethoxyphenoxy,
a glycoside of the L-xylose, wherein the aglycone moiety is 2-hydroxy-4-methylphenoxy,
a glycoside of the L-xylose, wherein the aglycone moiety is 2-hydroxy-4-ethylphenoxy,
a glycoside of the L-xylose, wherein the aglycone moiety is 5-(carboxymethyl)-2-hydroxyphenoxy,
a glycoside of the L-xylose, wherein the aglycone moiety is 5-(aminomethyl)-2-hydroxyphenoxy,
a glycoside of the L-xylose, wherein the aglycone moiety is 2-methoxy-4-methylphenoxy,
a glycoside of the L-xylose, wherein the aglycone moiety is 2-methoxy-4-ethylphenoxy,
a glycoside of the L-xylose, wherein the aglycone moiety is 4-(carboxymethyl)-2-methoxyphenoxy,
a glycoside of the L-xylose, wherein the aglycone moiety is 4-(aminomethyl)-2-methoxyphenoxy,
a glycoside of the L-xylose, wherein the aglycone moiety is 2-ethoxy-4-methylphenoxy,
a glycoside of the L-xylose, wherein the aglycone moiety is 2-ethoxy-4-ethylphenoxy,
a glycoside of the L-xylose, wherein the aglycone moiety is 4-(carboxymethyl)-2-ethoxyphenoxy,
a glycoside of the L-xylose, wherein the aglycone moiety is 4-(aminomethyl)-2-ethoxyphenoxy,
a glycoside of the L-arabinose, wherein the aglycone moiety is 2-hydroxy-5-methylphenoxy,
a glycoside of the L-arabinose, wherein the aglycone moiety is 2-hydroxy-5-ethylphenoxy,
a glycoside of the L-arabinose, wherein the aglycone moiety is 5-(carboxymethyl)-2-hydroxyphenoxy,
a glycoside of the L-arabinose, wherein the aglycone moiety is 5-(aminomethyl)-2-hydroxyphenoxy,
a glycoside of the L-arabinose, wherein the aglycone moiety is 2-methoxy-5-methylphenoxy,
a glycoside of the L-arabinose, wherein the aglycone moiety is 2-methoxy-5-ethylphenoxy,
a glycoside of the L-arabinose, wherein the aglycone moiety is 5-(carboxymethyl)-2-methoxyphenoxy,
a glycoside of the L-arabinose, wherein the aglycone moiety is 5-(aminomethyl)-2-methoxyphenoxy,
a glycoside of the L-arabinose, wherein the aglycone moiety is 2-ethoxy-5-methylphenoxy,
a glycoside of the L-arabinose, wherein the aglycone moiety is 2-ethoxy-5-ethylphenoxy,
a glycoside of the L-arabinose, wherein the aglycone moiety is 5-(carboxymethyl)-2-ethoxyphenoxy,
a glycoside of the L-arabinose, wherein the aglycone moiety is 5-(aminomethyl)-2-ethoxyphenoxy,
a glycoside of the L-arabinose, wherein the aglycone moiety is 2-hydroxy-4-methylphenoxy,
a glycoside of the L-arabinose, wherein the aglycone moiety is 2-hydroxy-4-ethylphenoxy,
a glycoside of the L-arabinose, wherein the aglycone moiety is 5-(carboxymethyl)-2-hydroxyphenoxy,
a glycoside of the L-arabinose, wherein the aglycone moiety is 5-(aminomethyl)-2-hydroxyphenoxy,
a glycoside of the L-arabinose, wherein the aglycone moiety is 2-methoxy-4-methylphenoxy,
a glycoside of the L-arabinose, wherein the aglycone moiety is 2-methoxy-4-ethylphenoxy,
a glycoside of the L-arabinose, wherein the aglycone moiety is 4-(carboxymethyl)-2-methoxyphenoxy,
a glycoside of the L-arabinose, wherein the aglycone moiety is 4-(aminomethyl)-2-methoxyphenoxy,
a glycoside of the L-arabinose, wherein the aglycone moiety is 2-ethoxy-4-methylphenoxy,
a glycoside of the L-arabinose, wherein the aglycone moiety is 2-ethoxy-4-ethylphenoxy,
a glycoside of the L-arabinose, wherein the aglycone moiety is 4-(carboxymethyl)-2-ethoxyphenoxy,
a glycoside of the L-arabinose, wherein the aglycone moiety is 4-(aminomethyl)-2-ethoxyphenoxy,
a glycoside of the L-mannose, wherein the aglycone moiety is 2-hydroxy-5-methylphenoxy,
a glycoside of the L-mannose, wherein the aglycone moiety is 2-hydroxy-5-ethylphenoxy,
a glycoside of the L-mannose, wherein the aglycone moiety is 5-(carboxymethyl)-2-hydroxyphenoxy,
a glycoside of the L-mannose, wherein the aglycone moiety is 5-(aminomethyl)-2-hydroxyphenoxy,
a glycoside of the L-mannose, wherein the aglycone moiety is 2-methoxy-5-methylphenoxy,
a glycoside of the L-mannose, wherein the aglycone moiety is 2-methoxy-5-ethylphenoxy,
a glycoside of the L-mannose, wherein the aglycone moiety is 5-(carboxymethyl)-2-methoxyphenoxy,
a glycoside of the L-mannose, wherein the aglycone moiety is 5-(aminomethyl)-2-methoxyphenoxy,
a glycoside of the L-mannose, wherein the aglycone moiety is 2-ethoxy-5-methylphenoxy,
a glycoside of the L-mannose, wherein the aglycone moiety is 2-ethoxy-5-ethylphenoxy,
a glycoside of the L-mannose, wherein the aglycone moiety is 5-(carboxymethyl)-2-ethoxyphenoxy,
a glycoside of the L-mannose, wherein the aglycone moiety is 5-(aminomethyl)-2-ethoxyphenoxy,
a glycoside of the L-mannose, wherein the aglycone moiety is 2-hydroxy-4-methylphenoxy,
a glycoside of the L-mannose, wherein the aglycone moiety is 2-hydroxy-4-ethylphenoxy,
a glycoside of the L-mannose, wherein the aglycone moiety is 5-(carboxymethyl)-2-hydroxyphenoxy,
a glycoside of the L-mannose, wherein the aglycone moiety is 5-(aminomethyl)-2-hydroxyphenoxy,
a glycoside of the L-mannose, wherein the aglycone moiety is 2-methoxy-4-methylphenoxy,
a glycoside of the L-mannose, wherein the aglycone moiety is 2-methoxy-4-ethylphenoxy,
a glycoside of the L-mannose, wherein the aglycone moiety is 4-(carboxymethyl)-2-methoxyphenoxy,
a glycoside of the L-mannose, wherein the aglycone moiety is 4-(aminomethyl)-2-methoxyphenoxy,
a glycoside of the L-mannose, wherein the aglycone moiety is 2-ethoxy-4-methylphenoxy,
a glycoside of the L-mannose, wherein the aglycone moiety is 2-ethoxy-4-ethylphenoxy,
a glycoside of the L-mannose, wherein the aglycone moiety is 4-(carboxymethyl)-2-ethoxyphenoxy,
a glycoside of the L-mannose, wherein the aglycone moiety is 4-(aminomethyl)-2-ethoxyphenoxy,
a glycoside of the L-glucose, wherein the aglycone moiety is 2-hydroxy-5-methylphenoxy,
a glycoside of the L-glucose, wherein the aglycone moiety is 2-hydroxy-5-ethylphenoxy,
a glycoside of the L-glucose, wherein the aglycone moiety is 5-(carboxymethyl)-2-hydroxyphenoxy,
a glycoside of the L-glucose, wherein the aglycone moiety is 5-(aminomethyl)-2-hydroxyphenoxy,
a glycoside of the L-glucose, wherein the aglycone moiety is 2-methoxy-5-methylphenoxy,
a glycoside of the L-glucose, wherein the aglycone moiety is 2-methoxy-5-ethylphenoxy,
a glycoside of the L-glucose, wherein the aglycone moiety is 5-(carboxymethyl)-2-methoxyphenoxy,
a glycoside of the L-glucose, wherein the aglycone moiety is 5-(aminomethyl)-2-methoxyphenoxy,
a glycoside of the L-glucose, wherein the aglycone moiety is 2-ethoxy-5-methylphenoxy,
a glycoside of the L-glucose, wherein the aglycone moiety is 2-ethoxy-5-ethylphenoxy,
a glycoside of the L-glucose, wherein the aglycone moiety is 5-(carboxymethyl)-2-ethoxyphenoxy,
a glycoside of the L-glucose, wherein the aglycone moiety is 5-(aminomethyl)-2-ethoxyphenoxy,
a glycoside of the L-glucose, wherein the aglycone moiety is 2-hydroxy-4-methylphenoxy,
a glycoside of the L-glucose, wherein the aglycone moiety is 2-hydroxy-4-ethylphenoxy,
a glycoside of the L-glucose, wherein the aglycone moiety is 5-(carboxymethyl)-2-hydroxyphenoxy,
a glycoside of the L-glucose, wherein the aglycone moiety is 5-(aminomethyl)-2-hydroxyphenoxy,
a glycoside of the L-glucose, wherein the aglycone moiety is 2-methoxy-4-methylphenoxy,
a glycoside of the L-glucose, wherein the aglycone moiety is 2-methoxy-4-ethylphenoxy,
a glycoside of the L-glucose, wherein the aglycone moiety is 4-(carboxymethyl)-2-methoxyphenoxy,
a glycoside of the L-glucose, wherein the aglycone moiety is 4-(aminomethyl)-2-methoxyphenoxy,
a glycoside of the L-glucose, wherein the aglycone moiety is 2-ethoxy-4-methylphenoxy,
a glycoside of the L-glucose, wherein the aglycone moiety is 2-ethoxy-4-ethylphenoxy,
a glycoside of the L-glucose, wherein the aglycone moiety is 4-(carboxymethyl)-2-ethoxyphenoxy,
a glycoside of the L-glucose, wherein the aglycone moiety is 4-(aminomethyl)-2-ethoxyphenoxy,
wherein each of the sugar moieties may be present in the aldehyde form, the furanose form, or the pyranose form.

7. A pharmaceutical composition for the prophylaxis or treatment of a disease of man or animal from the group consisting of peripheral and autonomic neuropathies, central nervous degenerative diseases, high blood pressure, arteriosclerosis, venous insufficiency, diabetes mellitus, osteoporosis, cataract, and photoaging of the skin, or for the use of such a compound for the prophylaxis or treatment of such a disease, containing a compound according to claim 1 and galenic excipients and/or carriers, prepared for oral administration, wherein the oral preparation preferably comprises a coat with a film or a capsule, the material of which is stable in the biological conditions in the stomach and in the small intestine, wherein the material of the coat preferably is selected from the group consisting of anionic copolymers based on methacrylic acid and methyl-methacrylate, such as eudragit, in particular eudragit S, galactomannan, in particular ethylated guaran (guar galactomannan), dextran and poly-galactomannan fatty acid esters, in particular esters with lauric acid, amylose, in particular crosslinked amylose, chitosan, crosslinked chondroitin, and pectin.

8. A method of making a pharmaceutical composition of claim 7, comprising the step of mixing said compound in a physiologically effective dosage with galenic excipients and carriers and prepared to a predetermined form of administration.

9. A use of a compound of claim 1 for making a pharmaceutical composition for the prophylaxis or treatment of a disease of man or animal from the group consisting of peripheral and autonomic neuropathies, central nervous degenerative diseases, high blood pressure, arteriosclerosis, venous insufficiency, diabetes mellitus, osteoporosis, cataract, and photoaging of the skin.

10. A method for the prophylaxis or treatment of a disease of man or animal from the group consisting of peripheral and autonomic neuropathies, central nervous degenerative diseases, high blood pressure, arteriosclerosis, venous insufficiency, diabetes mellitus, osteoporosis, cataract, and photoaging of the skin, wherein to an organism, which falls ill or is at risk of falling ill with the disease, is administered a pharmaceutical composition of claim 7 in a predetermined physiologically effective dosage.

11. A method of making a compound of claim 1,
wherein a sugar, monomer, dimer or oligomer, is reacted with a protective group compound, wherein OH groups of the sugar are protected,
wherein the sugar is reacted with a compound of the Formula III 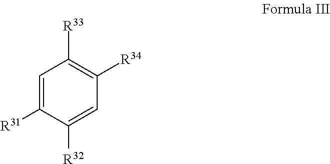
wherein R31 and R32, identical or different, are selected from —OH and —O—R35 with R35 being —H or —C1-C6 alkyl, linear or branched, saturated or not saturated, with the proviso that at least one of the moieties R31 or R32 is —OH, wherein R33 and R34, identical or different, may have the same meaning as R1 or is —CHO,
wherein the product of this reaction is freed from protective groups, wherein protected OH groups are reacted again to free OH groups, and
wherein before or after freeing protective groups, the product is optionally derivatized in the area of the moieties R33 and/or R34.
